(19) United States
(12) Patent Application Publication
(10) Pub. No.: US 2009/0226393 A1
(43) Pub. Date: Sep. 10, 2009

(54) POLYMER PACLITAXEL CONJUGATES AND METHODS FOR TREATING CANCER

(75) Inventors: Xinghe Wang, San Diego, CA (US); Gang Zhao, Vista, CA (US); Sang Van, San Diego, CA (US); Lei Yu, Carlsbad, CA (US)

Correspondence Address: KNOBBE MARTENS OLSON & BEAR LLP, 2040 MAIN STREET, FOURTEENTH FLOOR, IRVINE, CA, 92614, US

(73) Assignee: NITTO DENKO CORPORATION, OSAKA (JP)

(21) Appl. No.: 12/397,545

(22) Filed: Mar. 4, 2009

Related U.S. Application Data
(60) Provisional application No. 61/034,423, filed on Mar. 6, 2008.
(60) Provisional application No. 61/044,214, filed on Apr. 11, 2008.

(51) Int. Cl. A61K 47/48; A61P 35/00; A61K 31/337; A61K 31/74

(52) U.S. Cl. 424/78.17; 514/449

(57) ABSTRACT

Pharmaceutical compositions comprising a PGGA-PTX conjugate are prepared. The pharmaceutical compositions are used to treat a variety of cancers, such as lung cancer, skin cancer, kidney cancer, liver cancer and spleen cancer.

This application claims priority to U.S. Provisional Application No. 61/034,423, entitled “POLYMER CONJUGATES AND METHODS FOR TREATING CANCER,” filed on Mar. 6, 2008; and U.S. Provisional Application No. 61/044214, entitled “POLYMER CONJUGATES AND METHODS FOR TREATING CANCER,” filed on Apr. 11, 2008; both of which are incorporated herein by reference in their entireties for all purposes.

### BACKGROUND OF THE INVENTION

1. Field of the Invention

This invention relates generally to biocompatible polymer conjugates and methods of using them to treat cancer, and particularly to poly-(gamma-L-glutamyl glutamine)-paclitaxel and methods of using the polymer conjugate to treat cancer.

2. Description of the Related Art

A variety of systems have been used for the delivery of drugs, biomolecules, and imaging agents. For example, such systems include capsules, liposomes, microparticles, nanoparticles, and polymers.

A variety of polyester-based biodegradable systems have been characterized and studied. Polylactic acid (PLA), polyglycolic acid (PGA) and their copolymers polylactic-co-glycolic acid (PLGA) are some of the most well-characterized biomaterials with regard to design and performance for drug-delivery applications. See Uhrich, K. E.; Cannizzaro, S. M.; Langer, R. S. and Shakeshelf, K. M. “Polymeric Systems for Controlled Drug Release.” Chem. Rev. 1999, 99, 3181-3198 and Panyam J, Labhasetwar V. “Biodegradable nanoparticles for drug and gene delivery to cells and tissue.” Adv Drug Deliv Rev. 2003, 55, 329-47. Also, 2-hydroxypropyl methacrylate (HPMA) has been widely used to create a polymer for drug-delivery applications. Biodegradable systems based on polyorthoesters have also been investigated. See Heller, J.; Barr, J.; Ng, S. Y.; Abdellauoi, K. S. and Gurny, R. “Poly(ortho esters): synthesis, characterization, properties and uses.” Adv. Drug Del. Rev. 2002, 54, 1015-1039. Polyanhydride systems have also been investigated. Such polyanhydrides are typically biocompatible and may degrade in vivo into relatively non-toxic compounds that are eliminated from the body as metabolites. See Kumar, N.; Langer, R. S. and Domb, A. J. “Polyanhydrides: an overview.” Adv. Drug Del. Rev. 2002, 54, 889-91.

Amino acid-based polymers have also been considered as a potential source of new biomaterials. Poly-amino acids having good biocompatibility have been investigated to deliver low molecular-weight compounds. A relatively small number of polyglutamic acids and copolymers have been identified as candidate materials for drug delivery. See Bourke, S. L. and Kohn, J. “Polymers derived from the amino acid L-tyrosine: polycarbonates, polyarylates and copolymers with poly(ethylene glycol).” Adv. Drug Del. Rev., 2003, 55, 447-466.

Administered hydrophobic anticancer drugs and therapeutic proteins and polypeptides often suffer from poor bio-availability. In some cases it has been theorized that such poor bio-availability may be due to incompatibility of bi-phasic solutions of hydrophobic drugs and aqueous solutions and/or rapid removal of these molecules from blood circulation by enzymatic degradation. One technique that has been studied for increasing the efficacy of administered proteins and other small molecule agents entails conjugating the administered agent with a polymer, such as a polyethylene glycol (“PEG”) molecule, that can provide protection from enzymatic degradation in vivo. Such “PEGylation” often improves the circulation time and, hence, bio-availability of an administered agent.

PEG has shortcomings in certain respects, however. For example, because PEG is a linear polymer, the steric protection afforded by PEG is limited, as compared to branched polymers. Another shortcoming of PEG is that it is generally amenable to derivatization at its two terminals. This limits the number of other functional molecules (e.g. those helpful for protein or drug delivery to specific tissues) that can be readily conjugated to PEG.

Polyglutamic acid (PGA) is another polymer of choice for solubilizing hydrophobic anticancer drugs. Many anti-cancer drugs conjugated to PGA have been reported. See Chun Li. “Poly(L-glutamic acid)-anticancer drug conjugates.” Adv. Drug Del. Rev., 2002, 54, 695-713. However, none are currently FDA-approved.

Paclitaxel (PTX), extracted from the bark of the Pacific Yew tree (Wani et al. “Plant antitumor agents. VI. The isolation and structure of taxol, a novel antileukemic and antitumor agent from Taxus brevifolia.” J Am Chem Soc. 1971, 93, 2325-7), is a FDA-approved drug for the treatment of ovarian cancer and breast cancer. It is believed that pacilitaxel suffers from poor bio-availability. Approaches to improve bioavailability have been attempted, including formulating pacilitaxel in a mixture of Cremophor-EL and dehydrated ethanol (1:1, v/v) (Sparreboom et al. “Cremophor EL-mediated Alteration of Paclitaxel Distribution in Human Blood: Clinical Pharmacokinetic Implications.” Cancer Research 1999, 59, 1454-1457). This formulation is currently commercialized as Taxol™ (Bristol-Myers Squibb). However, this vehicle results in inadequate delivery of effective drug levels and high toxicity. The Taxol™ brand of paclitaxel has demonstrated clinical efficacy in non-small-cell lung cancer (NSCLC), but causes severe side effects including acute hypersensitivity reactions and peripheral neuropathies.

Another approach to improving paclitaxel bioavailability is by emulsification using high-shear homogenization (Constantinides et al. “Formulation Development and Antitumor Activity of a Filter-Sterilizable Emulsion of Paclitaxel.” Pharmaceutical Research 2000, 17, 175-182). Polymer-paclitaxel conjugates have been advanced in several clinical trials (Ruth Duncan “The Dawning era of polymer therapeutics.” Nature Reviews Drug Discovery 2003, 2, 347-360). Paclitaxel has been formulated into nano-particles with human albumin protein and has been used in clinical studies (Damascelli et al. “Intraarterial chemotherapy with polyoxyethylated castor oil free paclitaxel, incorporated in albumin nanoparticles (ABI-007): Phase II study of patients with squamous cell carcinoma of the head and neck and anal canal: preliminary evidence of clinical activity.” Cancer. 2001, 92, 2592-602, and Ibrahim et al. “Phase I and pharmacokinetic study of ABI-007, a Cremophor-free, protein-stabilized, nanoparticle formulation of paclitaxel.” Clin Cancer Res. 2002, 8, 1038-44). This formulation is currently commercialized as Abraxane® (American Pharmaceutical Partners, Inc.). However, existing formulations are not entirely satisfactory, and thus there is a long-felt need for improved paclitaxel formulations and methods of delivering them.

### SUMMARY OF THE INVENTION

Embodiments of polymer conjugates as described herein can be used to treat cancer. Methods for treating lung cancer, melanoma, kidney cancer, liver cancer and spleen cancer are provided in accordance with one aspect of the present invention. In some embodiments, a person suffering from cancer is identified and a polymer conjugate comprising poly-(gamma-L-glutamyl glutamine) (PGGA) and paclitaxel is administered to the person.

A pharmaceutical composition comprising a poly-(gamma-L-glutamyl glutamine)-paclitaxel polymer conjugate is provided in accordance with another aspect of the present invention. The molecular weight of the PGGA in the polymer conjugate is in the range of about 50,000 to about 100,000, and the weight percentage of paclitaxel in the polymer conjugate is in the range of about 20% to about 50%, based on total weight of the polymer conjugate.

These and other embodiments are described in greater detail below.

### BRIEF DESCRIPTION OF THE DRAWINGS

FIG. 1 shows a graph that illustrates the results of a plasma study comparing free paclitaxel (PTX) to poly-(gamma-L-glutamyl glutamine)-paclitaxel (MW=70k, weight percentage of paclitaxel in the polymer conjugate=35%) (PGGA_{70K}-PTX₃₅).

FIG. 2 shows a graph that illustrates the results of a tumor study in a NCI-460 human lung cancer model comparing free paclitaxel (PTX) to poly-(gamma-L-glutamyl glutamine)-paclitaxel (MW=70k, weight percentage of paclitaxel in the polymer conjugate=35%) (PGGA_{70K}-PTX₃₅).

FIG. 3 shows a graph that illustrates the results of a drug accumulation study in liver tissue comparing free paclitaxel (PTX) to poly-(gamma-L-glutamyl glutamine)-paclitaxel MW=70k, weight percentage of paclitaxel in the polymer conjugate=35%) (PGGA_{70K}-PTX₃₅).

FIG. 4 shows a graph that illustrates the results of a drug accumulation study in lung tissue comparing free paclitaxel (PTX) to poly-(gamma-L-glutamyl glutamine)-paclitaxel (MW=70k, weight percentage of paclitaxel in the polymer conjugate=35%) (PGGA_{70K}-PTX₃₅).

FIG. 5 shows a graph that illustrates the results of a drug accumulation study in spleen tissue comparing free paclitaxel (PTX) to poly-(gamma-L-glutamyl glutamine)-paclitaxel (MW=70k, weight percentage of paclitaxel in the polymer conjugate=35%) (PGGA_{70K}-PTX₃₅).

FIG. 6 shows a graph that illustrates the results of a drug accumulation study in kidney tissue comparing free paclitaxel (PTX) to poly-(gamma-L-glutamyl glutamine)-paclitaxel (MW=70k, weight percentage of paclitaxel in the polymer=35%) (PGGA_{70K}-PTX₃₅).

FIG. 7 shows a graph that illustrates the results of a drug accumulation study in muscles comparing free paclitaxel (PTX) to poly-(gamma-L-glutamyl glutamine)-paclitaxel (MW=70k, weight percentage of paclitaxel in the polymer=35%) (PGGA_{70K}-PTX₃₅).

FIG. 8 shows a bar graph that illustrates the percentage of free paclitaxel (PTX) and poly-(gamma-L-glutamyl glutamine)-paclitaxel (MW=70k, weight percentage of paclitaxel in the polymer conjugate=35%) (PGGA_{70K}-PTX₃₅) excreted by the kidneys within a 48 hour period.

FIG. 9 shows a bar graph that illustrates the percentage of free paclitaxel (PTX) and poly-(gamma-L-glutamyl glutamine)-paclitaxel (MW=70k, weight percentage of paclitaxel in the polymer conjugate=35%) (PGGA_{70K}-PTX₃₅) eliminated in feces within a 48 hour period.

FIG. 10 shows a graph that illustrates the anti-tumor activity of Abraxane® and poly-(gamma-L-glutamyl glutamine)-paclitaxel (MW=70k, weight percentage of paclitaxel in the polymer conjugate=35%) (PGGA_{70K}-PTX₃₅) in a B16 melanoma model.

FIG. 11 shows a graph that illustrates the percentage of body weight loss for Abraxane® and poly-(gamma-L-glutamyl glutamine)-paclitaxel (MW=70k, weight percentage of paclitaxel in the polymer conjugate=35%) (PGGA_{70K}-PTX₃₅) in a B16 melanoma model.

FIG. 12 shows a graph that illustrates the anti-tumor activity of Abraxane® and poly-(gamma-L-glutamyl glutamine)-paclitaxel (MW=70k, weight percentage of paclitaxel in the polymer conjugate=35%) (PGGA_{70K}-PTX₃₅) in a human non-small lung cancer model.

FIG. 13 shows a graph that illustrates the percentage of body weight loss for Abraxane® and poly-(gamma-L-glutamyl glutamine)-paclitaxel (MW=70k, weight percentage of paclitaxel in the polymer conjugate=35%) (PGGA_{70K}-PTX₃₅) in a human non-small lung cancer model.

FIG. 14 illustrates a reaction scheme for the preparation of poly-(γ-L-glutamyl glutamine).

FIG. 15 illustrates a general reaction scheme for the preparation of PGGA-PTX.

### DETAILED DESCRIPTION OF THE PREFERRED EMBODIMENTS

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The term “polymer conjugate” is used herein in its ordinary sense and thus includes polymers that are attached to one or more types of biologically active agent or drug, such as PTX. For example, PGGA-PTX is a polymer conjugate in which PGGA is attached to paclitaxel. The polymer (e.g., PGGA) may be attached directly to the other species (e.g., PTX) and may be attached by a linking group. The linking group may be a relatively small chemical moiety such as an ester or amide bond, or may be a larger chemical moiety, e.g., an alkyl ester linkage or an alkylene oxide linkage.

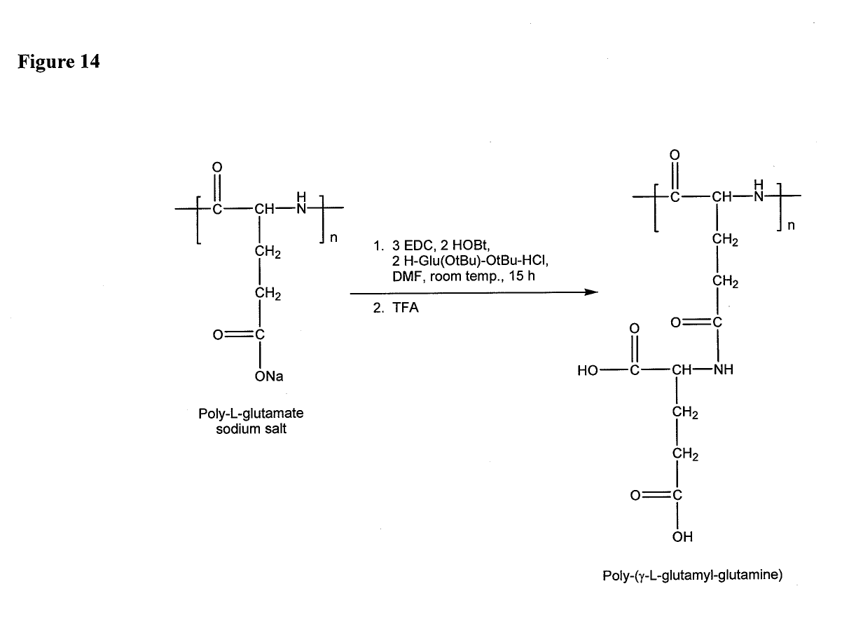

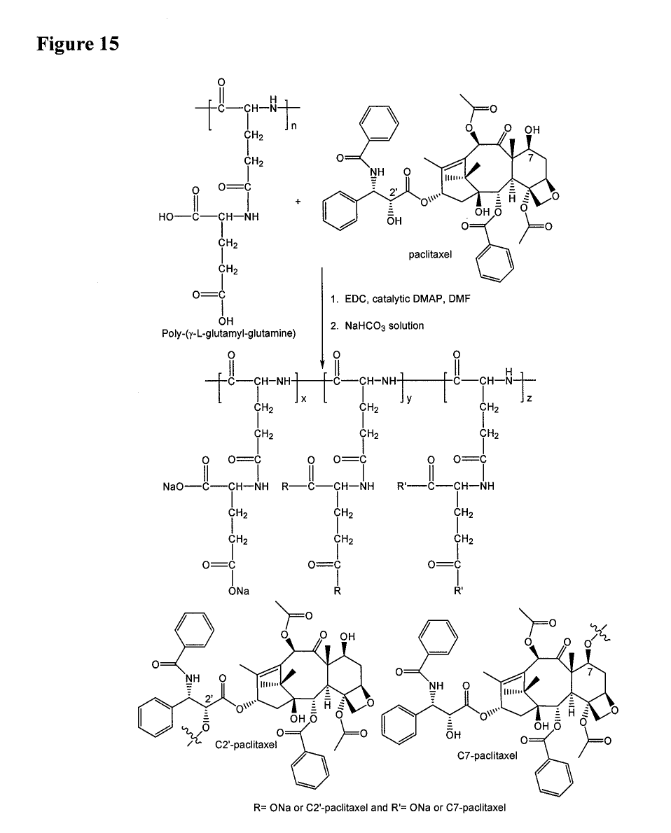

The term “polymer” is used herein in its ordinary sense and thus includes both homopolymers and copolymers having various molecular architectures. For example PGGA may be a homopolymer in which substantially all of the recurring units are gamma-L-glutamyl glutamine recurring units, or a copolymer in which most of the recurring units (e.g., more than 50 mole %, preferably more than 70 mole %, more preferably more than 90 mole %) are gamma-L-glutamyl glutamine recurring units. Some or all of the recurring units of the PGGA may be in the form of a salt, e.g., a sodium salt as illustrated in FIGS. 14-15. Thus reference herein to PGGA will be understood by those skilled in the art to include not only the acid form of PGGA but also forms of PGGA in which some or all of the recurring units are in a salt form.

Some embodiments provide a method of treating cancer using polymer conjugates. In general terms, such methods involve identifying a person who is suffering from a cancer selected from the group consisting of lung cancer, melanoma, kidney cancer, liver cancer and spleen cancer. Such identification may be by clinical diagnosis, e.g., involving known methods. In preferred embodiments, a polymer conjugate that comprises PGGA and paclitaxel, which may be referred to herein as PGGA-PTX, is administered to the person in an amount effective to treat the cancer. In certain embodiments, the molecular weight of the PGGA in the PGGA-PTX is in the range of about 50,000 to about 100,000 and the weight percentage of paclitaxel in the PGGA-PTX is in the range of about 20% to about 50%, based on total weight of PGGA-PTX. For example, in illustrated embodiments, the molecular weight of the PGGA is about 70,000, and/or the weight percentage of paclitaxel in the PGGA-PTX is about 35%.

Disclosed herein is a significant advance in cancer drug delivery technology. In an embodiment, the technology has the ability to overcome one or more of the aforementioned problems such as enhancing delivery of an anticancer agent. This invention is not bound by theory of operation, but is believed that the technology overcomes such problems through one or more mechanisms such as by enhanced permeability and/or retention mechanisms. One exemplary drug delivery composition includes PGGA-PTX in which the PGGA has a molecular weight of approximately 70,000 and the weight percentage of paclitaxel in the polymer conjugate is about 35%, which may be referred to herein as PGGA_{70K}-PTX₃₅. The PGGA-PTX compositions described herein can be made by conjugating PTX to PGGA, e.g., via ester bonds, e.g., as illustrated in FIGS. 14 and 15. Additional details for forming PGGA-PTX are described in U.S. Publication Serial No. 2007-0128118, entitled POLYGLUTAMATE-AMINO ACID CONJUGATES AND METHODS, which is hereby incorporated by reference in its entirety, and particularly for the purpose of describing such polymer conjugates and methods of making and using them. In some embodiments, PGGA-PTX spontaneously forms a nanoparticle in aqueous environments. PGGA-PTX compositions can be administered conveniently by intravenous injection.

A person suffering from cancer can be identified by techniques known in the art. For example, a person suffering from a particular cancer can identified by expression profiling of cancer marker genes that are known in the art. Expression profiling of tissue specific cancer marker genes can be performed using tissues that are obtained from lung tissue, skin tissue, kidney tissue, liver tissue and/or spleen tissue. Tissue specific cancer marker genes can be selected according to methods known in the art. In addition to, or instead of, using expression profiling, a person suffering from cancer can be identified using clinical methods and procedures known to those skilled in the art for diagnosing lung cancer, skin cancer, kidney cancer, liver cancer or spleen cancer.

The PGGA-PTX may administered through oral pathways or non-oral pathways, preferably non-oral. For example, in some embodiments, the PGGA-PTX is administered to the person by injection, e.g., intravenously. In some embodiments, the PGGA-PTX is administered locally to the lung, skin, kidney, liver and/or spleen.

In some embodiments, the PGGA-PTX per se is administered to a human patient. In other embodiments, the PGGA-PTX is administered in the form of pharmaceutical compositions in which the PGGA-PTX is mixed with at least one pharmaceutically suitable ingredient, such as a diluent, a suitable carrier and/or an excipient. For example, the pharmaceutical composition may be provided in the form of an injectable liquid.

The therapeutically effective amount of the PGGA-PTX suitable for a particular patient depends on the characteristics of the patient, the stage of advancement of the cancer and the type of cancer the patient suffers from. If the patient has been diagnosed as suffering from lung cancer, kidney cancer, liver cancer and/or spleen cancer, the PGGA-PTX may be advantageously administered to the person at a dose in the range of about 40 mg PTX equivalents/kg to about 550 mg PTX equivalents/kg. If the patient has been diagnosed as suffering from melanoma, the PGGA-PTX may be advantageously administered to the person at a dose in the range of about 40 mg PTX equivalents/kg to about 345 mg PTX equivalents/kg.

In some embodiments, a pharmaceutical composition comprising PGGA-PTX is provided. It has been found that the molecular weight of the PGGA and the amount of PTX in the PGGA-PTX influence the delivery characteristics and hence the efficacy of the PGGA-PTX. The molecular weight of the PGGA in the PGGA-PTX is preferably in the range of about 50,000 to about 100,000 and the weight percentage of paclitaxel in the PGGA-PTX is preferably in the range of about 20% to about 50%, based on total weight of the PGGA-PTX. In some embodiments, the molecular weight of the PGGA is about 70,000. In other embodiments, the weight percentage of paclitaxel in the PGGA-PTX is about 35%. In yet other embodiments, the molecular weight of the PGGA is about 70,000, and the weight percentage of paclitaxel in the PGGA-PTX is about 35%.

### Pharmaceutical Compositions

The term “pharmaceutical composition” refers to a mixture of a compound disclosed herein (e.g., PGGA-PTX) with other chemical components, such as diluents, excipients and/or carriers. The pharmaceutical composition facilitates administration of the compound to an organism. Multiple techniques of administering a compound exist in the art including, but not limited to, oral, injection, aerosol, parenteral, and topical administration.

The term “carrier” refers to a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” refers to chemical compounds diluted in water that will dissolve the compound of interest (e.g., PGGA-PTX) as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound. The term “physiologically acceptable” refers to a carrier or diluent that does not abrogate the biological activity and properties of the compound.

In some embodiments, prodrugs, metabolites, stereoisomers, hydrates, solvates, polymorphs, and pharmaceutically acceptable salts of the compounds disclosed herein (e.g., the polymer conjugate and/or the agent that it comprises) are provided.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid, phosphoric acid and the like. Pharmaceutical salts can also be obtained by reacting a compound with an organic acid such as aliphatic or aromatic carboxylic or sulfonic acids, for example acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine, lysine, and the like.

If the manufacture of pharmaceutical formulations involves intimate mixing of the pharmaceutical excipients and the active ingredient in its salt form, then it may be desirable to use pharmaceutical excipients which are non-basic, that is, either acidic or neutral excipients.

In various embodiments, the compounds disclosed herein (e.g., PGGA-PTX) can be used alone, in combination with other compounds disclosed herein, or in combination with one or more other agents active in the therapeutic areas described herein.

In another aspect, the present disclosure relates to a pharmaceutical composition comprising one or more physiologically acceptable surface active agents, carriers, diluents, excipients, smoothing agents, suspension agents, film forming substances, and coating assistants, or a combination thereof; and a compound (e.g., PGGA-PTX) disclosed herein. Acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium methasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The PGGA-PTX per se described herein can be administered to a human patient or in pharmaceutical compositions in which the PGGA-PTX is mixed with other active ingredients, as in combination therapy, or suitable carriers or excipients. Techniques for formulation and administration may be found in “Remington's Pharmaceutical Sciences,” Mack Publishing Co., Easton, Pa., 18th edition, 1990.

Suitable routes of administration may, for example, include oral, rectal, transmucosal, topical, or intestinal administration; parenteral delivery, including intramuscular, subcutaneous, intravenous, intramedullary injections, as well as intrathecal, direct intraventricular, intraperitoneal, intranasal, or intraocular injections. The compounds (e.g., PGGA-PTX) can also be administered in sustained or controlled release dosage forms, including depot injections, osmotic pumps, pills, transdermal (including electrotransport) patches, and the like, for prolonged and/or timed, pulsed administration at a predetermined rate.

The pharmaceutical compositions described herein may be manufactured in a manner that is itself known, e.g., by means of conventional mixing, dissolving, granulating, dragee-making, levigating, emulsifying, encapsulating, entrapping or tabletting processes. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized. For transmucosal administration, penetrants appropriate to the barrier to be permeated may be used in the formulation. Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the compounds can be formulated readily by combining the active compounds (e.g., PGGA-PTX) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Further disclosed herein are various pharmaceutical compositions well known in the pharmaceutical art for uses that include intraocular, intranasal, and intraauricular delivery. Suitable penetrants for these uses are generally known in the art. Pharmaceutical compositions for intraocular delivery include aqueous ophthalmic solutions of the active compounds in water-soluble form, such as eyedrops, or in gellan gum (Shedden et al., Clin. Ther., 23(3):440-50 (2001)) or hydrogels (Mayer et al., Ophthalmologica, 210(2):101-3 (1996)); ophthalmic ointments; ophthalmic suspensions, such as microparticulates, drug-containing small polymeric particles that are suspended in a liquid carrier medium (Joshi, A., J. Ocul. Pharmacol., 10(l):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts. All of the above-mentioned references, are incorporated herein by reference in their entireties. Such suitable pharmaceutical formulations are most often and preferably formulated to be sterile, isotonic and buffered for stability and comfort. Pharmaceutical compositions for intranasal delivery may also include drops and sprays often prepared to simulate in many respects nasal secretions to ensure maintenance of normal ciliary action. As disclosed in Remington's Pharmaceutical Sciences, 18th Ed., Mack Publishing Co., Easton, Pa. (1990), which is incorporated herein by reference in its entirety, and well-known to those skilled in the art, suitable formulations are most often and preferably isotonic, slightly buffered to maintain a pH of 5.5 to 6.5, and most often and preferably include antimicrobial preservatives and appropriate drug stabilizers. Pharmaceutical formulations for intraauricular delivery include suspensions and ointments for topical application in the ear. Common solvents for such aural formulations include glycerin and water.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For hydrophobic compounds, a suitable pharmaceutical carrier may be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few hours or weeks up to over 100 days. Depending on the chemical nature and the biological stability of the therapeutic reagent, additional strategies for protein stabilization may be employed.

Agents intended to be administered intracellularly may be administered using techniques well known to those of ordinary skill in the art. For example, such agents may be encapsulated into liposomes. All molecules present in an aqueous solution at the time of liposome formation are incorporated into the aqueous interior. The liposomal contents are both protected from the external micro-environment and, because liposomes fuse with cell membranes, are efficiently delivered into the cell cytoplasm. The liposome may be coated with a tissue-specific antibody. The liposomes will be targeted to and taken up selectively by the desired organ. Alternatively, small hydrophobic organic molecules may be directly administered intracellularly.

Additional therapeutic or diagnostic agents may be incorporated into the pharmaceutical compositions. Alternatively or additionally, pharmaceutical compositions may be combined with other compositions that contain other therapeutic or diagnostic agents.

### Methods of Administration

The compounds or pharmaceutical compositions may be administered to the patient by any suitable means. Non-limiting examples of methods of administration include, among others, (a) administration though oral pathways, which administration includes administration in capsule, tablet, granule, spray, syrup, or other such forms; (b) administration through non-oral pathways such as rectal, vaginal, intraurethral, intraocular, intranasal, or intraauricular, which administration includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; as well as (e) administration topically; as deemed appropriate by those of skill in the art for bringing the active compound into contact with living tissue.

Pharmaceutical compositions suitable for administration include compositions where the active ingredients (e.g., PTX) are contained in an amount effective to achieve its intended purpose. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

As will be readily apparent to one skilled in the art, the useful in vivo dosage to be administered and the particular mode of administration will vary depending upon the age, weight and mammalian species treated, the particular compounds employed, and the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

In non-human animal studies, applications of potential products are commenced at higher dosage levels, with dosage being decreased until the desired effect is no longer achieved or adverse side effects disappear. The dosage may range broadly, depending upon the desired effects and the therapeutic indication. Typically, dosages may be between about 10 ug/kg and 100 mg/kg body weight, preferably between about 100 ug/kg and 10 mg/kg body weight. Alternatively dosages may be based and calculated upon the surface area of the patient, as understood by those of skill in the art.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, which is hereby incorporated herein by reference in its entirety, with particular reference to Ch. 1, p. 1). Typically, the dose range of the composition administered to the patient can be from about 0.5 to 1000 mg/kg of the patient's body weight. The dosage may be a single one or a series of two or more given in the course of one or more days, as is needed by the patient. In instances where human dosages for compounds have been established for at least some condition, the present invention will use those same dosages, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Although the exact dosage will be determined on a drug-by-drug basis, in most cases, some generalizations regarding the dosage can be made. The daily dosage regimen for an adult human patient may be, for example, an oral dose of between 0.1 mg and 2000 mg of each active ingredient, preferably between 1 mg and 500 mg, e.g. 5 to 200 mg. In other embodiments, an intravenous, subcutaneous, or intramuscular dose of each active ingredient of between 0.01 mg and 100 mg, preferably between 0.1 mg and 60 mg, e.g. 1 to 40 mg is used. In cases of administration of a pharmaceutically acceptable salt, dosages may be calculated as the free base. In some embodiments, the composition is administered 1 to 4 times per day. Alternatively the compositions of the invention may be administered by continuous intravenous infusion, preferably at a dose of each active ingredient up to 1000 mg per day. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections. In some embodiments, the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered may be dependent on the subject being treated, on the subject's weight, the severity of the affliction, the manner of administration and the judgment of the prescribing physician.

Compounds disclosed herein (e.g., the polymer conjugate and/or the agent that it comprises) can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. Similarly, acceptable animal models may be used to establish efficacy of chemicals to treat such conditions. When selecting a model to determine efficacy, the skilled artisan can be guided by the state of the art to choose an appropriate model, dose, and route of administration, and regime. Of course, human clinical trials can also be used to determine the efficacy of a compound in humans.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

Dosage amounts may be adjusted based on the maximum tolerated dose (MTD) of the pharmaceutical composition. For example, the MTD of PGGA-PTX can be evaluated in tumor free and tumored nude mice. The therapeutic efficacy of PGGA-PTX can be evaluated in a xenograft model of human NSCLC (NCI-H460) and compared to Abraxane®. Preferred formulations of PGGA-PTX are readily soluble in saline (50 mg/ml). As illustrated in the Examples below, treatment with multiple injections of PGGA_{70K}-PTX₃₅ (q7dx2, i.v.) demonstrated superior antitumor activity compared to Abraxane® at their respective MTDs or corresponding dose levels (P=0.008). Additionally, PGGA_{70K}-PTX₃₅ caused a 136% tumor growth delay (TGD) compared to Abraxane®. These observations indicate that PGGA-PTX (preferably having a PGGA molecular weight in the range of about 50,000 to about 100,000 and a PTX weight percentage in the range of about 20% to about 50%) can provide a solution to the toxicity problems encountered with other anticancer drug delivery systems. Furthermore, PGGA-PTX can allow for the delivery of a higher dosage of the drug in animals which can lead to superior anticancer therapeutic efficacies.

In the Examples below, [³ H]PGGA₇₀ₖ-[³H]PTX₃₅ was administered as an intravenous bolus injection to mice bearing subcutaneous NCI-H460 lung cancer xenografts at a dose of 40 mg PTX equivalents/kg. Plasma, tumor and samples of the major organ were collected at intervals out to 340 hours. [³H]-PTX in plasma and digested tissue samples was quantified by liquid scintillation counting. Pharmacokinetic parameters were estimated using WinNonlin software using a non-compartment model.

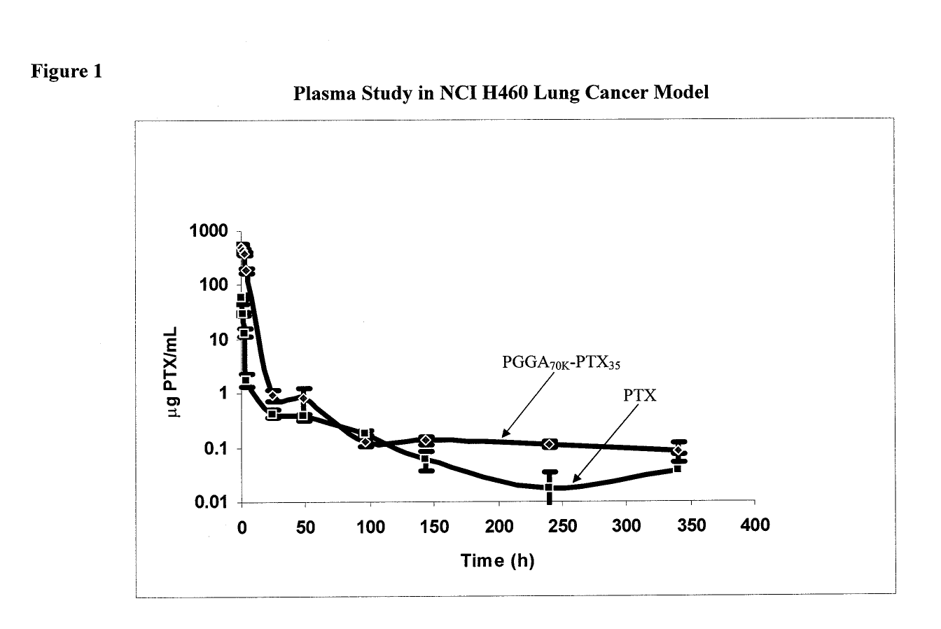

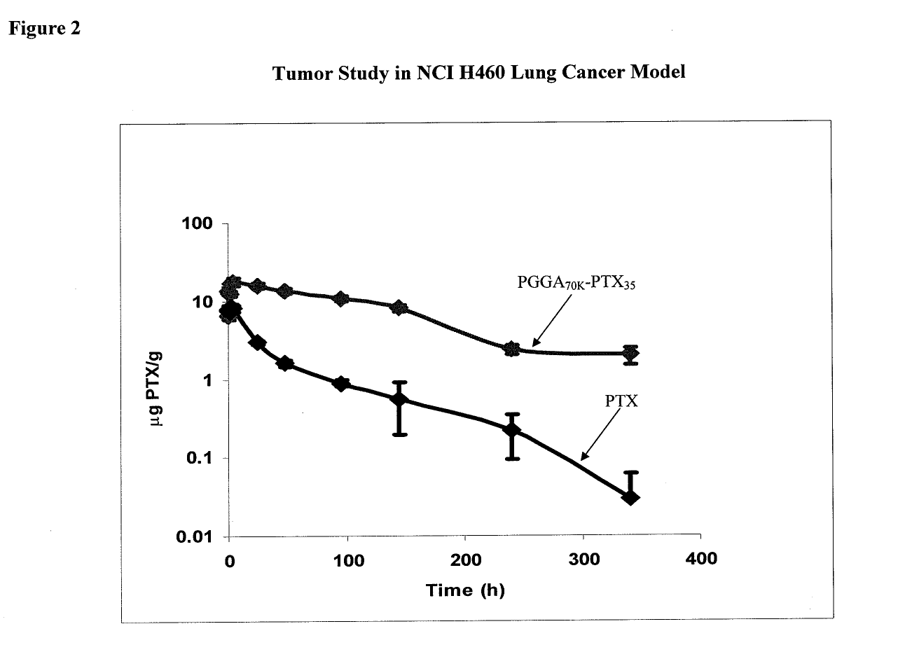

FIGS. 1 and 2 are graphs that illustrate the results of plasma and tumor studies, respectively, comparing PGGA_{70K}-PTX₃₅ to free paclitaxel (PTX). In plasma the AUCₗₐₛₜ for [³H]PGGA_{70K}-PTX₃₅ and [³H]PTX was 3,454 and 146 μg-h/ml, respectively, while the Cₘₐₓ values were 517 and 60 μg/ml, respectively. Thus, at equivalent PTX doses, use of PGGA_{70K}-PTX₃₅ increased the AUCₗₐₛₜ by a factor of 23.6-fold and the Cₘₐₓ by 8.5-fold. The mean terminal half-life of [³H]PGGA_{70K}-PTX₃₅ was 296 hours whereas that for [³H]PTX was 59.9 hours. Additionally, both [³H]PGGA_{70K}-PTX₃₅ and [³H]PTX were rapidly distributed to well-perfused tissues. In tumor tissue the AUCₗₐₛₜ for [³H] PGGA_{70K}-PTX₃₅ and [³H]PTX was 2,496 and 323 μg-h/ml, respectively, while the Cₘₐₓ values were 17 and 8.3 μg/ml. Thus, at equivalent PTX doses use of PGGA_{70K}-PTX₃₅ increased AUCₗₐₛₜ in the tumor by a factor of 7.7-fold and the Cₘₐₓ by 2.1-fold. The terminal half-lives of [³H]PGGA_{70K}-PTX₃₅ and [³H]PTX in the tumor tissue were 107 and 51 hours, respectively. Additionally, the volume of distribution of [³H]PGGA_{70K}-PTX₃₅ and [³H]PTX were 48976 and 23167 mL/kg, respectively. Tables 1 and 2 summarize the plasma and tumor pharmacokinetics for [³H]PGGA_{70K}-PTX₃₅ and [³H]PTX.

TABLE 1
Plasma Pharmacokinetics

| | | | T_{1/2} | CL | |
|---|---|---|---|---|---|
| | Cₘₐₓ | AUCₗₐₛₜ | Terminal | (ml/h/ | Vd |
| | (ng/ml) | (ng hr/ml) | (hr) | kg) | (ml/kg) |
| [³H]PTX | 60533 | 146265 | 60 | 267 | 23167 |
| (40 mg/kg PTX) | | | | | |
| Total PTX | | | | | |
| PGGA-[³H]PTX | 517084 | 3454375 | 296 | 11.5 | 48976 |
| (PTX 40 mg/kg) | | | | | |
| Total PTX | | | | | |
| PGGA-[³H]PTX/ | 8.5 | 24 | 5 | 0.04 | 0.2 |
| [³H]PTX Ratio | | | | | |

TABLE 2
Tumor Pharmacokinetics

| | | | T_{1/2} | CL | |
|---|---|---|---|---|---|
| | Cₘₐₓ | AUCₗₐₛₜ | Terminal | (ml/h/ | Tₘₐₓ |
| | (ng/ml) | (ng hr/ml) | (hr) | kg) | (h) |
| [³H]PTX | 8327 | 322589 | 51 | 123 | 2 |
| (40 mg/kg PTX) | | | | | |
| Total PTX | | | | | |
| PGGA-[³H]PTX | 17538 | 2496055 | 107 | 14.65 | 4 |
| (PTX 40 mg/kg) | | | | | |
| Total PTX | | | | | |
| PGGA-[³H]PTX/ | 2 | 8 | 2 | 0.12 | 2 |
| [³H]PTX Ratio | | | | | |

The ability of PGGA_{70K}-PTX₃₅ to provide increased delivery of PTX to tumors was associated with a substantial increase in anti-tumor activity and therapeutic index in the NCI-H460 lung cancer xenograft model. Furthermore, incorporation of PTX into the PGGA_{70K}-PTX₃₅ polymer significantly prolonged the half-life of PTX in both the plasma and tumor compartments. This resulted in a 7.7-fold increase in the amount of PTX delivered to the tumor, and this was associated with a substantial increase in efficacy as measured by tumor growth delay.

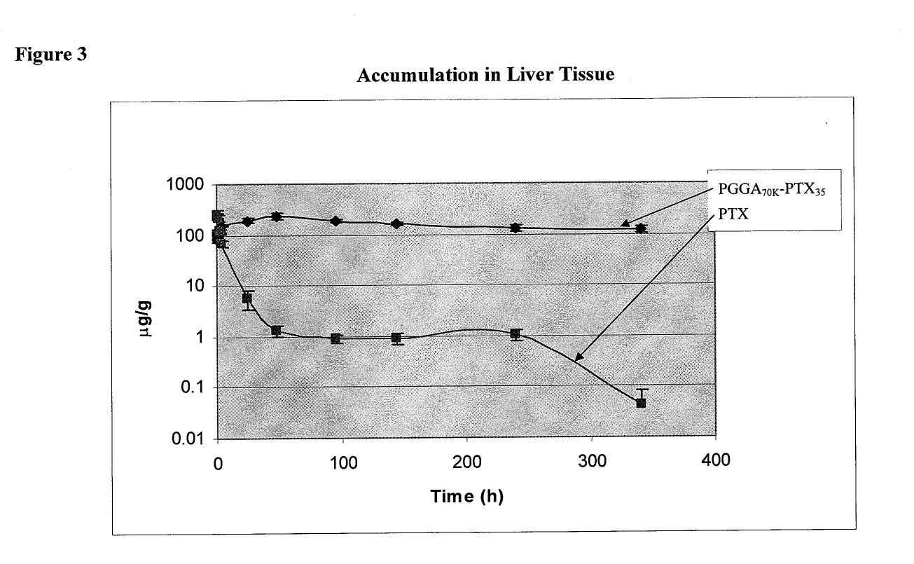

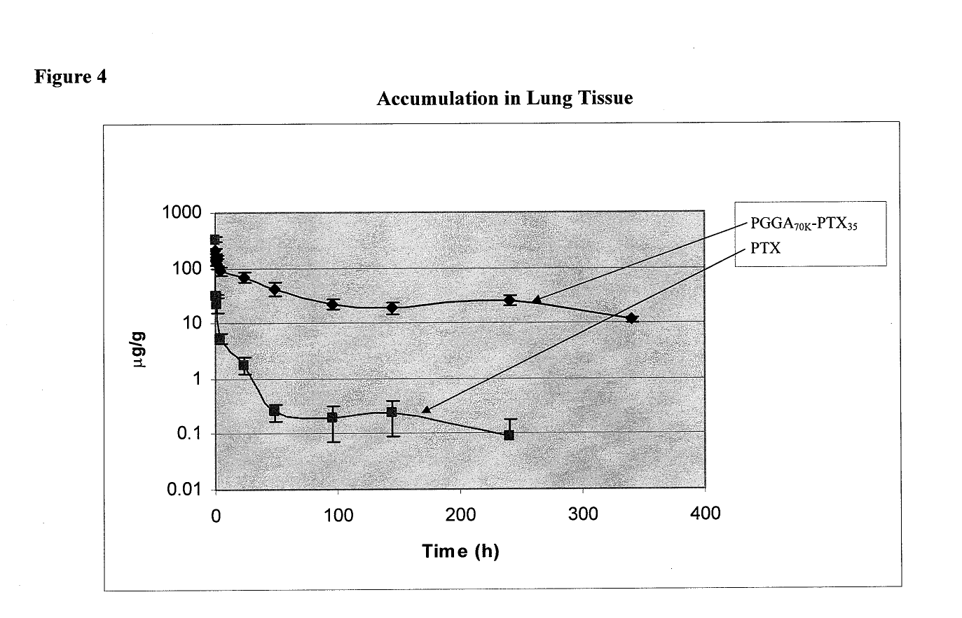

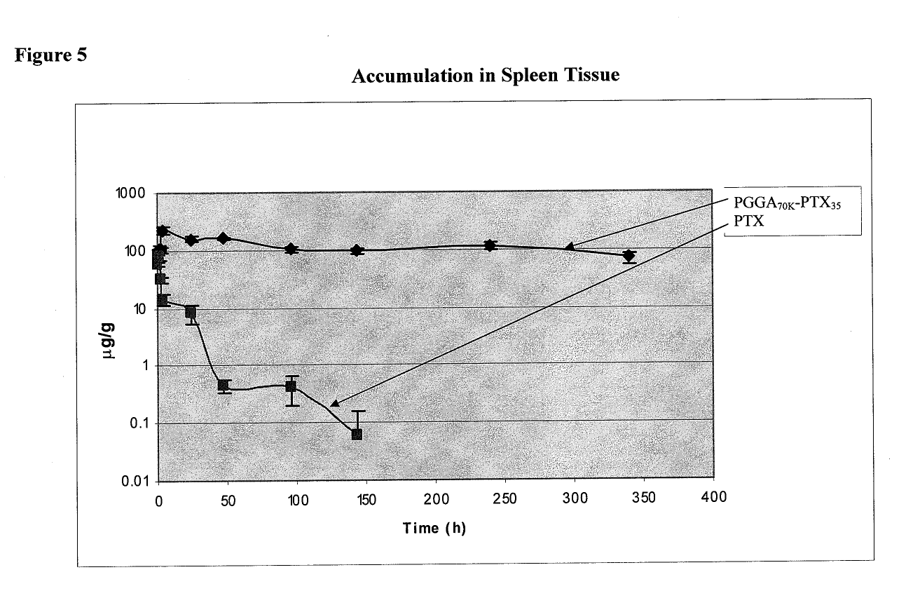

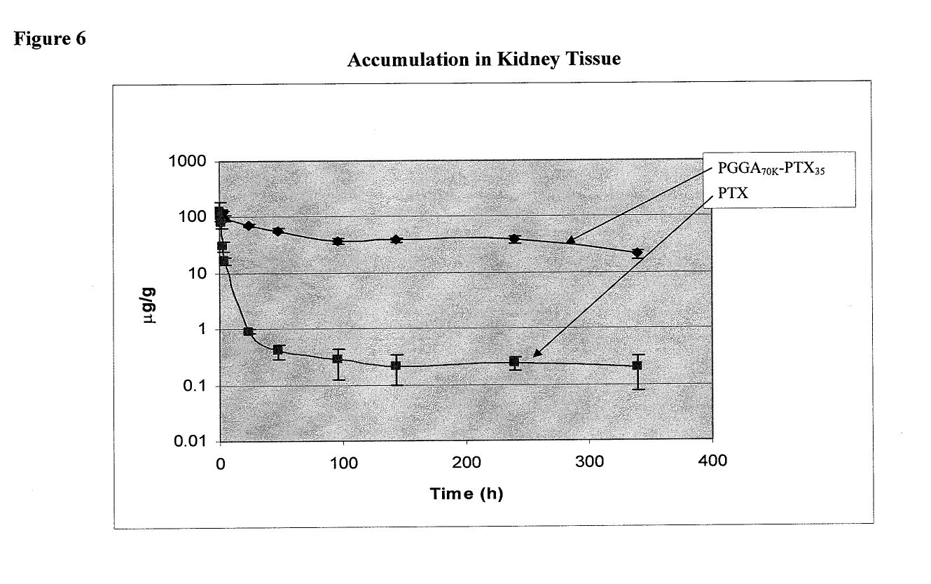

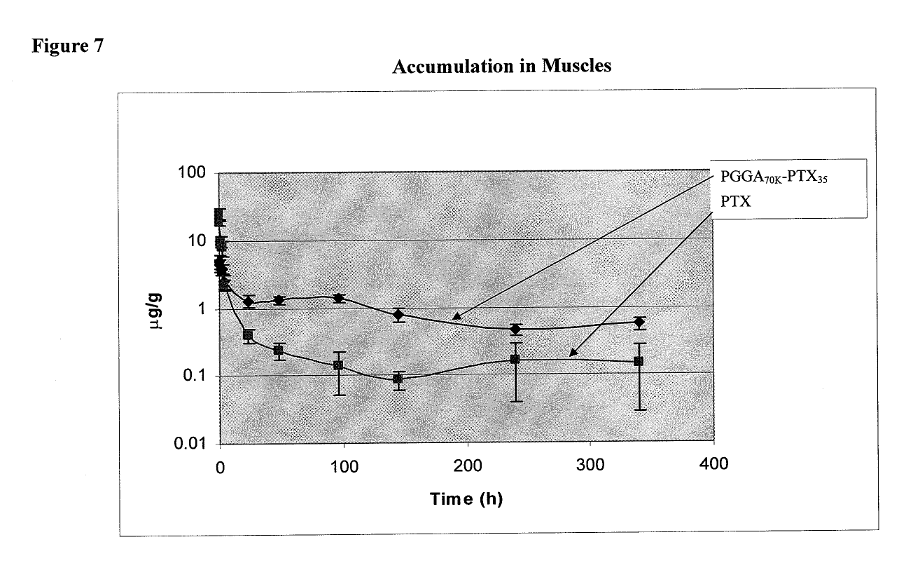

FIGS. 3-7 and Table 3 provide the results of a drug accumulation study in various organs for PGGA_{70K}-PTX₃₅ and PTX. PGGA_{70K}-PTX₃₅ is much more stable in liver, lung, kidney and spleen. A significant amount of PGGA_{70K}-PTX₃₅ was retained in the above-mentioned organs 48 hours post administration. For example, 48 hours post administration, there remained about 230 μg/g PGGA_{70K}-PTX₃₅ in liver, 40 μg/g PGGA_{70K}-PTX₃₅ in lung, 60 μg/g PGGA_{70K}-PTX₃₅ in kidney, and 160 μg/g PGGA_{70K}-PTX₃₅ in spleen. In contrast, there was a much lower amount of free PTX retained in the above-mentioned organs 48 hours after administration. In all the above-mentioned organs, there were less than 2 μg/g PTX 48 hours after administration. The results indicate that PGGA_{70K}-PTX₃₅ is a more effective anti-cancer drug than free PTX in liver, lung, kidney and spleen.

TABLE 3
Biodistribution in Different Organs

| | Tissue | 2 h | 4 h | 24 h | 48 h | 144 h |
|---|---|---|---|---|---|---|
| PGGA_{70K}- | Blood | 371.44 ± 26.36 | 191.49 ± 19.46 | 0.94 ± 0.21 | 0.82 ± 0.46 | 0.13 ± 0.022 |
| PTX₃₅ | Tumor | 16.99 ± 1.51 | 17.54 ± 1.99 | 15.66 ± 1.21 | 13.58 ± 0.93 | 8.05 ± 0.84 |
| | Liver | 122.86 ± 9.59 | 154.94 ± 3.89 | 192.99 ± 21.51 | 230.79 ± 29.38 | 165.78 ± 11.38 |
| | Lung | 137.91 ± 29.72 | 90.04 ± 17.49 | 70.62 ± 13.66 | 42.55 ± 12.09 | 19.37 ± 4.48 |
| | Kidney | 119.36 ± 13.69 | 98.63 ± 13.14 | 71.32 ± 5.83 | 56.73 ± 5.16 | 38.38 ± 4.12 |
| | Spleen | 102.48 ± 11.42 | 223.28 ± 27.96 | 160.09 ± 18.66 | 161.01 ± 8.61 | 96.23 ± 13.24 |
| | Muscle | 3.8315 ± 0.62 | 2.49 ± 0.60 | 1.29 ± 0.26 | 1.32 ± 0.18 | 0.79 ± 0.19 |
| PTX | Blood | 13.38 ± 2.39 | 1.79 ± 0.47 | 0.42 ± 0.07 | 0.37 ± 0.053 | 0.059 ± 0.023 |
| | Tumor | 8.33 ± 0.70 | 8.13 ± 0.78 | 2.95 ± 0.20 | 1.61 ± 0.15 | 0.31 ± 0.19 |
| | Liver | 116.75 ± 11.79 | 66.81 ± 8.70 | 5.67 ± 2.22 | 1.29 ± 0.31 | 0.92 ± 0.230 |
| | Lung | 22.23 ± 6.25 | 5.42 ± 1.06 | 1.79 ± 0.61 | 0.25 ± 0.087 | 0.24 ± 0.16 |
| | Kidney | 31.03 ± 6.62 | 16.6 ± 2.63 | 0.92 ± 0.098 | 0.421 ± 0.12 | 0.22 ± 0.12 |
| | Spleen | 31.44 ± 4.34 | 14.47 ± 3.27 | 8.42 ± 3.10 | 0.45 ± 0.13 | 0.062 ± 0.10 |
| | Muscle | 7.86 ± 2.10 | 2.22 ± 0.39 | 0.41 ± 0.094 | 0.24 ± 0.065 | 0.086 ± 0.027 |

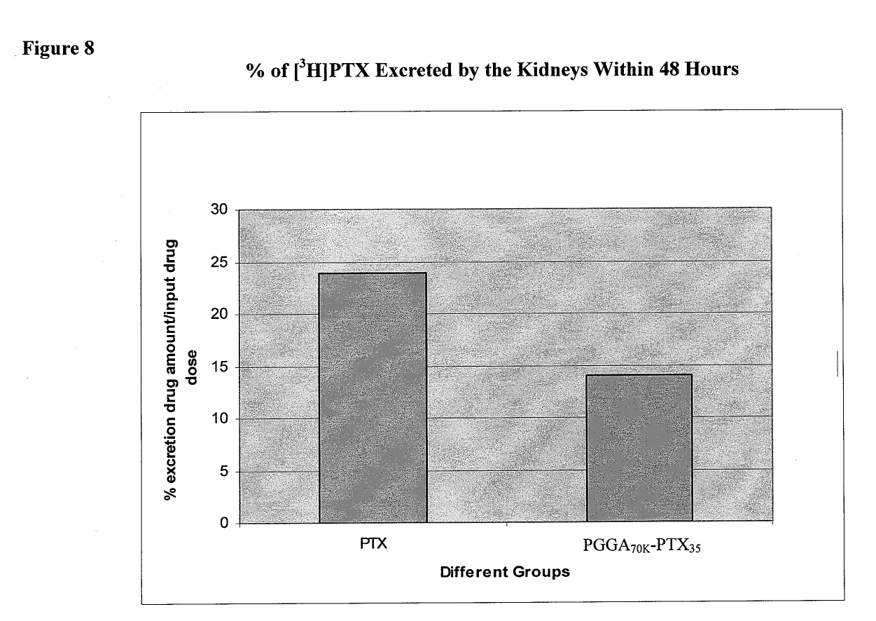

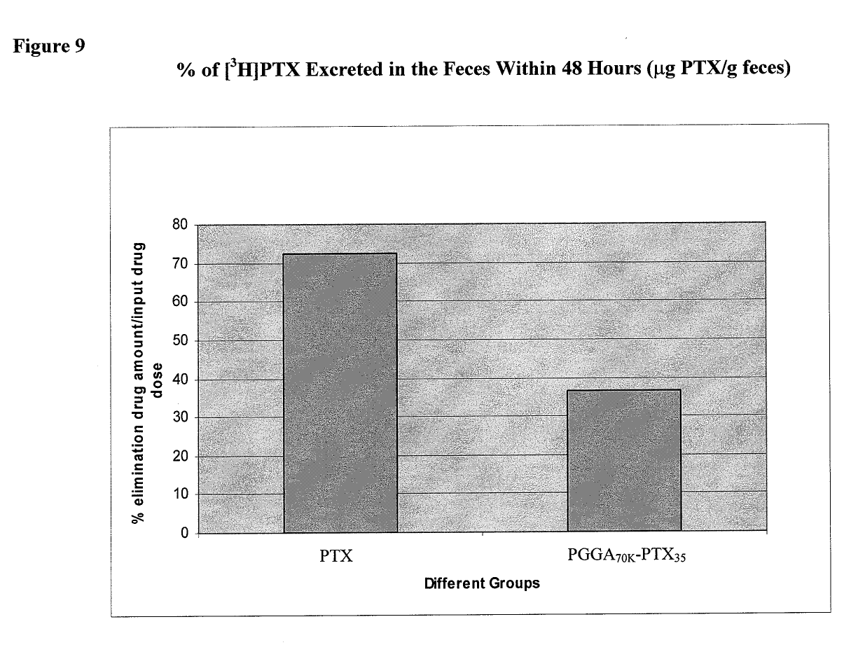

FIGS. 8 and 9 are bar graphs that illustrate the percentage of PGGA_{70K}-PTX₃₅ and free paclitaxel (PTX) excreted by the kidneys within a 48 hour period and eliminated in feces within a 48 hour period, respectively, As shown by FIGS. 8 and 9, PGGA_{70K}-PTX₃₅ was degraded after injection and excreted by kidney (urine). The estimated total urinary excretion in a 48 hour period was 23.5% for PTX and 13.9% for PGGA_{70K}-PTX₃₅. A substantial fraction of the administered dose was recovered in the feces for both PGGA_{70K}-PTX₃₅ and PTX. In mice injected with ³[H]-PTX, approximately 72% of the compound was detected in the feces within the first 48 hour. By comparison, for mice injected with [³H]PGGA_{70K}-PTX₃₅, only 36% of the composition was detected in the feces in the same the 48 hour time period. The results indicate a greater amount of the drug from PGGA_{70K}-PTX₃₅ stays in the body compared to PTX in a given time period. These results are consistent with the biodistribution results discussed above, and further confirm that PGGA_{70K}-PTX₃₅ is a more effective anti-cancer drug than PTX in liver, lung, kidney and spleen. Moreover, these results indicate that PGGA_{70K}-PTX₃₅ can be degraded in the circulation and whole body system.

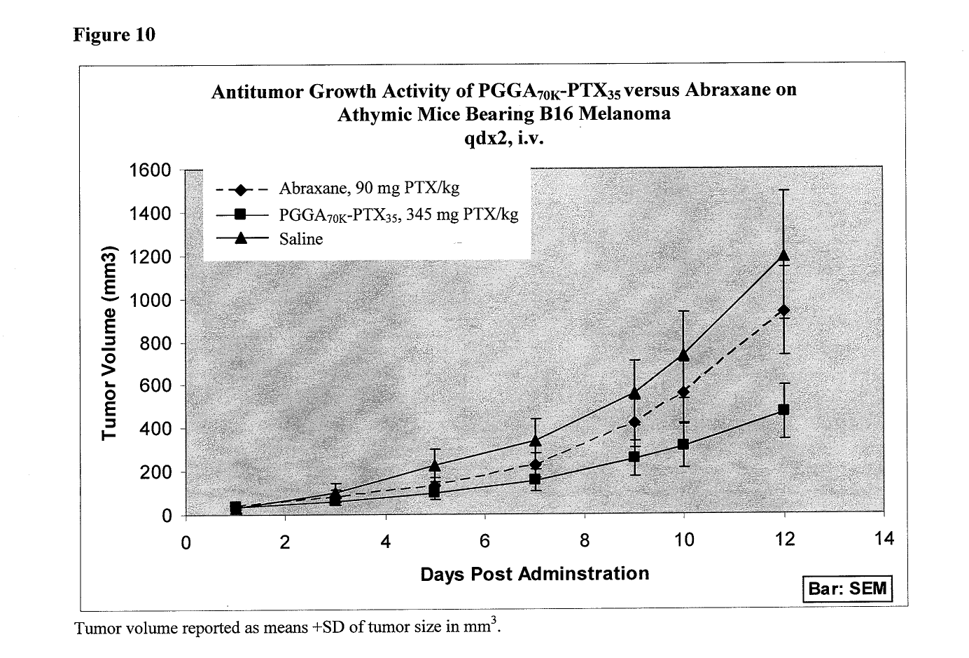

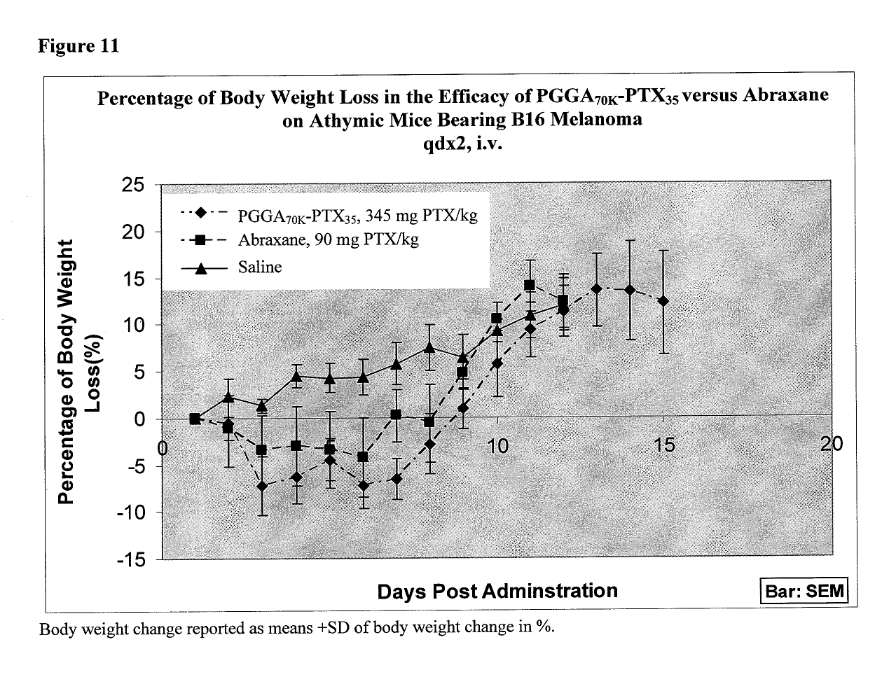

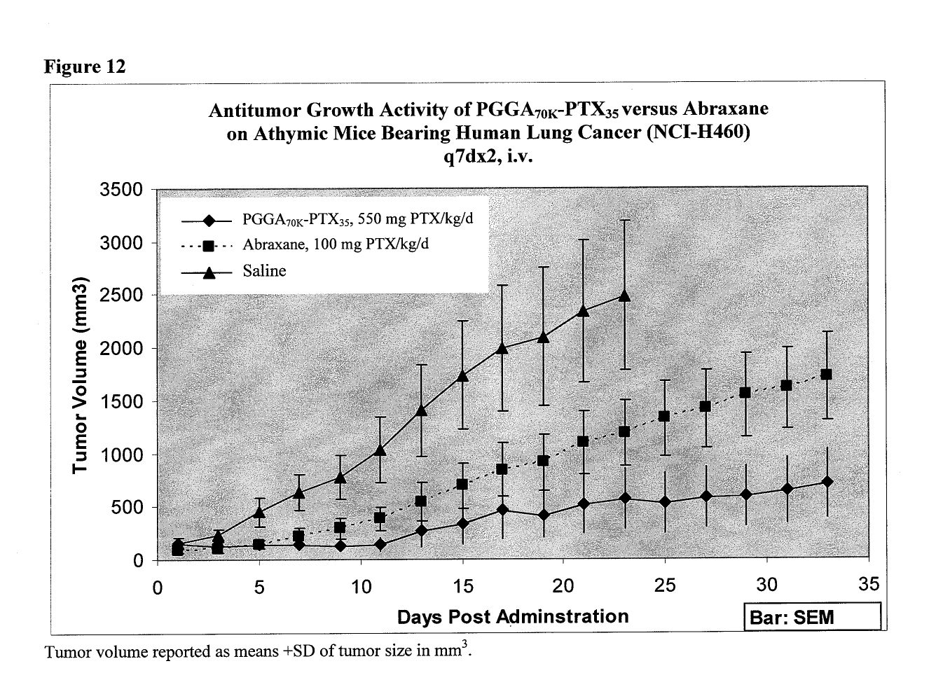

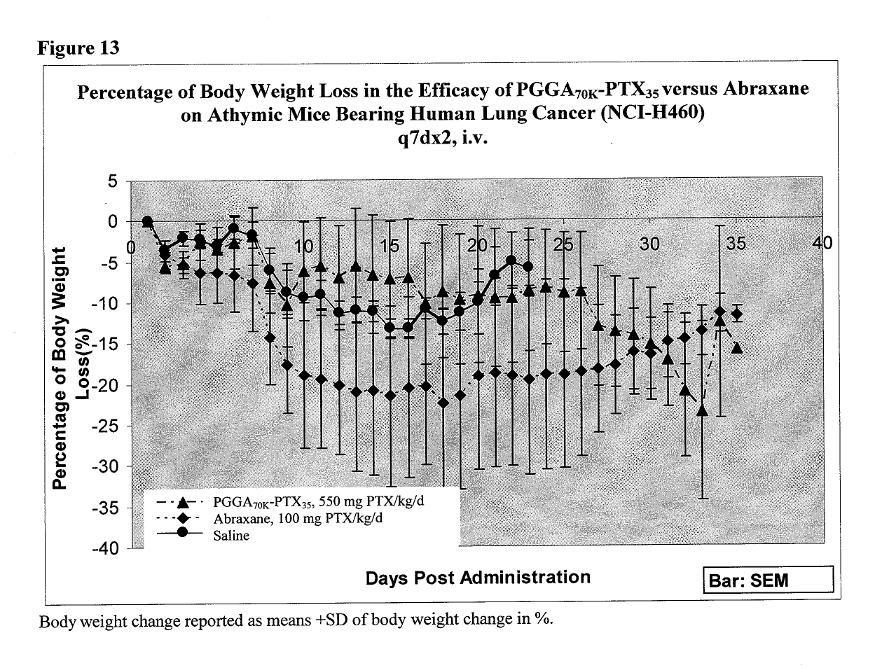

FIG. 10 compares the antitumor growth activity of PGGA_{70K}-PTX₃₅ versus Abraxane® against B16 melanoma. Mice that were subject to PGGA_{70K}-PTX₃₅ administration have significantly reduced tumor volume comparing to mice subjected to Abraxane® administration. FIG. 11 compares the toxicity of PGGA_{70K}-PTX₃₅ to Abraxane® and shows that PGGA_{70K}-PTX₃₅ and Abraxane® have similar toxicity to mice as indicated by the percentage of body weight loss. FIGS. 12 and 13 show the comparison results of antitumor activity and toxicity between PGGA_{70K}-PTX₃₅ and Abraxane® in mice with lung cancer. As shown in the figures, PGGA_{70K}-PTX₃₅ has stronger antitumor activity than Abraxane®. These results indicate that PGGA_{70K}-PTX₃₅ is a better antitumor drug than Abraxane®.

### EXAMPLES

The following examples are provided for the purposes of further describing the embodiments described herein, and do not limit the scope of the invention.

### Materials:

Poly-L-glutamate sodium salts with different molecular weights (average molecular weights of 41,400 (PGA(97k)), 17,600 (PGA(44k)), 16,000 (PGA(32k)), and 10,900 (PGA(21k)) daltons based on multi-angle light scattering (MALS)); 1,3-dicyclohexyl carbodiimide (DCC); N-(3-dimethylaminopropyl)-N′-ethylcarbodiimide hydrochloride (EDC); hydroxybenzotriazole (HOBt); pyridine; 4-dimethylaminopyridine (DMAP); N,N′-dimethylformamide (DMF); gadolinium-acetate; chloroform; and sodium bicarbonate were purchased from Sigma-Aldrich Chemical company. Poly-L-glutamate was converted into poly-L-glutamic acid using 2 N hydrochloric acid solution. Trifluoroacetic acid (TFA) was purchased from Bioscience. Omniscan™ (gadodiamide) was purchased from GE healthcare.

¹H NMR was obtained from Joel (400 MHz), and particle sizes were measured by ZetalPals (Brookhaven Instruments Corporation). Microwave chemistry was carried out in a Biotage instrument. Molecular weights of polymers were determined by size exclusion chromatography (SEC) combined with a multi-angle light scattering (MALS) (Wyatt Corporation) detector:

SEC-MALS Analysis Conditions:

| | |
|---|---|
| HPLC system: | Agilent 1200 |
| Column: | Shodex SB 806M HQ |
| | (exclusion limit for Pullulan is 20,000,000, |
| | particle size: 13 micron, size (mm) |
| | ID × Length; 8.0 × 300) |
| Mobile Phase: | 1 × DPBS or 1% LiBr in DPBS (pH 7.0) |
| Flow Rate: | 1 ml/min |
| MALS detector: | DAWN HELEOS from Wyatt |
| DRI detector: | Optilab rEX from Wyatt |
| On-line Viscometer: | ViscoStar from Wyatt |
| Software: | ASTRA 5.1.9 from Wyatt |
| Sample Concentration: | 1-2 mg/ml |
| Injection volume: | 100 μl |

dn/dc value of polymer: 0.185 was used in the measurement.
BSA was used as a control before actual samples are run.

Using the system and conditions described above (hereinafter, referred to as the Heleos system with MALS detector), the average molecular weight of the starting polymers (poly-L-glutamate sodium salts average molecular weights of 41,400, 17,600, 16,000, and 10,900 daltons reported by Sigma-Aldrich using their system with MALS) were experimentally found to be 49,000, 19,800, 19,450, and 9,400 daltons, respectively,.

The content of paclitaxel in polymer-paclitaxel conjugates was estimated by UV/Vis spectrometry (Lambda Bio 40, PerkinElmer) based on a standard curve generated with known concentrations of paclitaxel in methanol (λ=228 nm).

### Example 1

PGGA-PTX was prepared according to the general scheme illustrated in FIGS. 14 and 15.

First, a poly-(γ-L-glutamyl-glutamine) was prepared according to the general scheme illustrated in FIG. 14.

Polyglutamate sodium salt (0.40 g) having an average molecular weight of 19,800 daltons based on the Heleos system with MALS detector, EDC (1.60 g), HOBt (0.72 g), and H-glu(OtBu)-(OtBu)-HCl (1.51 g) were mixed in DMF (30 mL). The reaction mixture was stirred at room temperature for 15-24 hours and then was poured into distilled water solution (200 mL). A white precipitate formed and was filtered and washed with water. The intermediate polymer was then freeze-dried. The intermediate polymer structure was confirmed via ¹H-NMR by the presence of a peak for the O-tBu group at 1.4 ppm.

The intermediate polymer was treated with TFA (20 mL) for 5-8 hours. The TFA was then partially removed by rotary evaporation. Water was added to the residue and the residue was dialyzed using semi-membrane cellulose (molecular weight cut-off 10,000 daltons) in reverse-osmosis water (4 time water changes) overnight. Poly-(γ-L-glutamyl-glutamine) was transparent at pH 7 in water after dialysis. Poly-(γ-L-glutamyl-glutamine) (0.6 g) was obtained as white powder after being lyophized. The polymer structure was confirmed via ¹H-NMR by the disappearance of the peak for the O-tBu group at 1.4 ppm. The average molecular weight of poly-(γ-L-glutamyl-glutamine) was measured and found to be 38,390 daltons.

PGGA-PTX was then prepared according to the general scheme illustrated in FIG. 15

Poly-(γ-L-glutamyl-glutamine)-average molecular weight of 110,800 daltons (1.0 g was partially dissolved in DMF (55 mL). EDC (600 mg) and paclitaxel (282 mg) were added, respectively, into the mixture. DMAP (300 mg), acting as a catalyst, was added into the mixture. The reaction mixture was stirred at room temperature for 1 day. Completion of the reaction was verified by TLC. The mixture was poured into diluted 0.2N hydrochloric acid solution (300 mL). A precipitate formed and was collected after centrifugation at 10,000 rpm. The residue was then re-dissolved in sodium bicarbonate solution 0.5 M NaHCO₃ solution. The polymer solution was dialyzed in deionized water using a cellulose membrane (cut-off 10,000 daltons) in reverse-osmosis water (4 time water changes) for 1 day. A clear solution was obtained and freeze-dried. PGGA-PTX (1.1 g) was obtained and confirmed by ¹H NMR. The content of paclitaxel in PGGA-PTX was determined by UV spectrometry as 20% by weight to weight.

### Example 2

### Pharmacokinetics

Female, nu/nu mice were inoculated SC with 4×106 human lung cancer NCI-H460 cells grown in tissue culture on each shoulder and each hip (4×107 cells/mL in RPMI1640 medium, injection volume 0.1 ml). At the point when the mean tumor volume for the entire population had reached 400-500 mm3 (9-10 mm diameter), each mouse received a single IV bolus injection of ³H-labelled PTX or PGGA-[³H]PTX. The dose for both [³H]PTX and PGGA-[³H]PTX was 40 mg PTX equivalents/kg. For each drug, groups of 6 mice were anesthetized at various time points and 0.3 ml of blood, obtained by cardiac puncture, was collected into heparinized tubes. Thereafter, mice were sacrificed before recovering from anesthesia and the following tissues were harvested and frozen from each animal: each of the 4 tumors, lung, liver, spleen, both kidneys, skeletal muscle and heart. Mice were sacrificed at the following times after the end of the IV bolus injection: 0 (i.e. as quickly as possible after the IV injection), 0.166, 0.5, 1, 2, 4, 24, 48, 96, 144, 240 and 340 h. For each drug a total 72 mice were required (6 mice/time point, 12 time points).

### Example 3

### Cancer Studies

PGGA_{70K}-PTX₃₅ was readily soluble in saline (50 mg/ml). The maximum tolerated dose (MTD) of PGGA_{70K}-PTX₃₅ was evaluated in tumor free and tumor nude mice (Charles River, Mass.), and therapeutic efficacy of PGGA_{70K}-PTX₃₅ as compared to Abraxane (ABI, CA) was evaluated in both NCI-H460 non-small cell lung cancer xenograft and murine B16 melanoma model. Antitumor growth activity of PGGA70K-PTX35 and the toxicity of PGGA70K-PTX35 to Athymic mice bearing B16 melonoma or human lung cancer are shown in Tables 4 and 5, and FIGS. 10-13.

TABLE 4
Melanoma

| | | Paclitaxel | | | |
|---|---|---|---|---|---|
| | | Equivalent | | | |
| Agent | n | (mg/kg) | Route | Schedule | % TGD |
| Saline | 3 | N/A | IV | qdx2 | N/A |
| Abraxane ® | 3 | 90 | IV | qdx2 | |
| PGGA_{70K}-PTX₃₅ | 3 | 345 | IV | qdx2 | 50 |

TABLE 5
Non-small cell lung cancer

| | | Paclitaxel | | | |
|---|---|---|---|---|---|
| | | Equivalent | | | |
| Agent | n | (mg/kg) | Route | Schedule | % TGD |
| Saline | 2 | N/A | IV | q7dx2 | N/A |
| Abraxane ® | 3 | 100 | IV | q7dx2 | |
| PGGA_{70K}-PTX₃₅ | 2 | 550 | IV | q7dx2 | 136 |

It will be understood by those of skill in the art that numerous and various modifications can be made without departing from the spirit of the present invention. Therefore, it should be clearly understood that the forms of the present invention are illustrative only and not intended to limit the scope of the present invention.

What is claimed is:

1. A method of treating cancer, comprising:
identifying a person suffering from a cancer selected from the group consisting of lung cancer, melanoma, kidney cancer, liver cancer and spleen cancer; and
administering a polymer conjugate to the person in an amount effective to treat the cancer;
wherein the polymer conjugate comprises poly-(gamma-L-glutamyl glutamine) (PGGA) and paclitaxel (PTX);
wherein the molecular weight of the PGGA is in the range of about 50,000 to about 100,000; and
wherein the weight percentage of paclitaxel in the polymer conjugate is in the range of about 20% to about 50%, based on total weight of the polymer conjugate.

2. The method of claim 1, wherein the molecular weight of the PGGA is about 70,000.

3. The method of claim 1, wherein the weight percentage of paclitaxel in the polymer conjugate is about 35%.

4. The method of claim 1, wherein the molecular weight of the PGGA in the polymer conjugate is about 70,000, and the weight percentage of paclitaxel in the polymer conjugate is about 35%.

5. The method of claim 1, wherein the polymer conjugate is administered to the person by injection.

6. The method of claim 1, wherein the polymer conjugate is administered locally to the lung, skin, kidney or spleen.

7. The method of claim 1, wherein the polymer conjugate is administered in a mixture with at least one pharmaceutically suitable ingredient selected from a diluent, a carrier and an excipient.

8. The method of claim 1, wherein the person has been diagnosed as suffering from melanoma and the polymer conjugate is administered to the person at a dose in the range of about 40 mg PTX equivalents/kg to about 345 mg PTX equivalents/kg.

9. The method of claim 1, wherein the person has been diagnosed as suffering from at least one selected from the group consisting of lung cancer, kidney cancer, liver cancer and spleen cancer, and wherein the polymer conjugate is administered to the person at a dose in the range of about 40 mg PTX equivalents/kg to about 550 mg PTX equivalents/kg.

10. The method of claim 1, wherein the person suffering from the cancer has been identified by expression profiling of cancer marker genes obtained from at least one tissue selected from the group consisting of lung tissue, skin tissue, kidney tissue, liver tissue and spleen tissue.

11. A pharmaceutical composition comprising a poly-(gamma-L-glutamyl glutamine) (PGGA) and paclitaxel (PTX) polymer conjugate, wherein the molecular weight of the PGGA is in the range of about 50,000 to about 100,000, and wherein the weight percentage of PTX in the polymer conjugate is in the range of about 20% to about 50%, based on total weight of the polymer conjugate.

12. The pharmaceutical composition of claim 11, wherein the weight percentage of PTX in the polymer conjugate is about 35%.

13. The pharmaceutical composition of claim 12, wherein the molecular weight of the PGGA is about 70,000.

14. A pharmaceutical composition comprising the polymer conjugate of claim 11 and at least one pharmaceutically acceptable ingredient selected from an excipient, a carrier, and a diluent.

15. The pharmaceutical composition of claim 14, in the form of an injectable liquid.
